(19) United States
(12) Patent Application Publication
(10) Pub. No.: US 2026/0325863 A1
(43) Pub. Date: Oct. 22, 2026

(54) PHOTOTHERAPY DEVICE

(71) Applicant: DONGGUAN LAIGUANG ELECTRONIC TECHNOLOGY CO., LTD., Dongguan City (CN)

(72) Inventor: JIANGXIANG WU, Dongguan City (CN)

(73) Assignee: DONGGUAN LAIGUANG ELECTRONIC TECHNOLOGY CO., LTD., Dongguan City (CN)

(21) Appl. No.: 19/529,079

(22) Filed: Feb. 3, 2026

(30) Foreign Application Priority Data
Dec. 31, 2025 (CN) 202522822601.7

(51) Int. Cl. A61N 5/06

(52) U.S. Cl. CPC A61N 5/06; A61N 2005/0633; A61N 2005/0645

(57) ABSTRACT

The present application provides a phototherapy device, which includes a phototherapy component and a support base. The phototherapy component is provided with a phototherapy module and a mounting module, and the phototherapy module is configured to output corresponding phototherapy light. The mounting module is provided on a rear surface of the phototherapy component and is provided with a first docking member. The support base is provided with a support main body, a deformable member and a second docking member. The deformable member is connected to the support main body and can deform under external force. The second docking member is connected to an end of the deformable member away from the support main body and can rotate relative to the end of the deformable member away from the support main body. The second docking member is detachably docked with the first docking member.

### CROSS-REFERENCE TO RELATED APPLICATIONS

The present application claims priority to Chinese patent application No. 202522822601.7, filed on Dec. 31, 2025, the entire contents of which are incorporated herein by reference.

### TECHNICAL FIELD

The present application relates to the technical field of phototherapy devices, and in particular, to a phototherapy device.

### BACKGROUND

With the development of science and technology, phototherapy devices have been widely applied in people's daily lives. The light emitted by phototherapy devices is used for physical therapy on users, so as to achieve a therapeutic effect, improve skin conditions, promote blood circulation and enhance skin nutrient supply.

In the related art, conventional phototherapy devices consist of a fixed support and a phototherapy component, where the phototherapy component faces the user and is connected to the fixed support. However, the phototherapy component is in a fixed state and can only maintain a single position without the capability of deformation adjustment, which results in poor scenario compatibility of the conventional phototherapy devices.

### SUMMARY

An object of the present application is to provide a phototherapy device, which includes a phototherapy component and a support base. The phototherapy component is provided with a phototherapy module and a mounting module. The phototherapy module is provided on a front surface of the phototherapy component for outputting corresponding phototherapy light. The mounting module is provided on a rear surface of the phototherapy component and provided with a first docking member. The support base is provided with a support main body, a deformable member and a second docking member. The deformable member is connected to the support main body and can deform under external force. The second docking member is connected to an end of the deformable member away from the support main body and can rotate relative to the end of the deformable member away from the support main body. The second docking member is detachably docked with the first docking member, and the phototherapy component can rotate along with the rotation of the second docking member. Thus, phototherapy component can be deformed through the deformable member; meanwhile, the phototherapy component can adjust its position in different directions during rotation, enabling compatibility with multiple different scenarios, thereby avoiding that the phototherapy component can only maintain a single state and cannot be deformed, and improving the scenario compatibility of the phototherapy device.

In order to achieve above purpose, the present application provides a following technical solution: a phototherapy device, including:
a phototherapy component, provided with a phototherapy module and a mounting module; and
a support base, provided with a support main body, a deformable member and a second docking member;
wherein the phototherapy module is provided on a front surface of the phototherapy component for outputting corresponding phototherapy light;
the mounting module is provided on a rear surface of the phototherapy component and provided with a first docking member;
the deformable member is connected to the support main body and is deformable under external force;
the second docking member is connected to and is rotatable relative to an end of the deformable member away from the support main body; and
the second docking member is detachably docked with the first docking member, and the phototherapy component is rotatable along with a rotation of the second docking member.

In one embodiment, the first docking member is provided with a first docking portion;
the second docking member is provided with a second docking portion, and the second docking portion is snap-fit with the first docking portion; and
the second docking member is rotatable relative to the end of the deformable member away from the support main body.

In one embodiment, the second docking member is provided with a rotation hole;
the end of the deformable member away from the support main body is inserted into the rotation hole, and the second docking member is rotatable along an axis of the rotation hole and is configured to drive the phototherapy component to rotate through the first docking member.

In one embodiment, the deformable member is provided with a deformable main body and a swing member;
the deformable main body is connected to the support main body and located on one side of the support main body;
the swing member is swingably connected to the deformable main body and is configured to swing horizontally;
a front surface of the swing member is provided with the second docking member, and the swing member is connected to the phototherapy component through the second docking member and the first docking member; and
a horizontal position of the phototherapy component is adjusted along with a horizontal swinging of the swing member relative to the deformable main body.

In one embodiment, the swing member is hinged to the deformable main body and is configured to swing horizontally along a hinged position; and
the deformable main body is provided with a deformable tube inside, the deformable tube is deformable under external force, and an outer surface of the deformable main body is a colloid layer.

In one embodiment, the rear surface of the phototherapy component is provided with a containing groove;
the first docking member is telescopically connected to the rear surface of the phototherapy component and is telescopical relative to the containing groove;
the second docking member is inserted into the containing groove and is in a docking state with the first docking member in the containing groove; and
the second docking member is inserted into a snap-fit groove of the first docking member and is in snap-fit with the first docking member.

In one embodiment, the rear surface of the phototherapy component is provided with a button;
the button is movably connected to the phototherapy component and is telescopical relative to the rear surface of the phototherapy component; and
the button is connected to the first docking member and is configured to drive the first docking member to telescope relative to the containing groove during movement.

In one embodiment, the rear surface of the phototherapy component is provided with a moving groove;
the first docking member is provided with a first inclined portion and a snap-fit portion, and the first inclined portion and the snap-fit portion are a unitary one-piece structure;
the button is provided with a moving main body, a pressing portion and a second inclined portion;
the moving main body is movably connected to the moving groove;
the pressing portion is connected to the moving main body and located on a rear side wall of the button, and the pressing portion serves as a pressing position of the button;
the second inclined portion is connected to the moving main body and docked with the first inclined portion; and
the first inclined portion is configured to contact different positions of the second inclined portion during a movement of the second inclined portion, and drive the snap-fit portion to telescope relative to the containing groove, so that the snap-fit portion is in snap-fit with or separated from the second docking member.

In one embodiment, the support main body is provided with a base and a lifting portion;
the base is configured for being laid on the ground;
the lifting portion is liftably connected to the base and is configured for supporting the deformable member; and
the lifting portion is configured to penetrate through the base, lift relative to the base along a vertical direction, and is fixed on the base through a tightening member to maintain a corresponding height.

In one embodiment, the phototherapy component is provided with a phototherapy main body which supports the phototherapy module and the mounting module;
a plurality of phototherapy modules are provided along a length direction of the phototherapy main body; and
two adjacent phototherapy modules are in a planar state or an angled state.

In one embodiment, the phototherapy main body is provided with a plurality of through holes;
each through hole is configured for a connection belt to pass through, and the connection belt is configured to pass through a corresponding through hole and wound around a user's body portion.

### BRIEF DESCRIPTIONS OF THE DRAWINGS

To describe the technical solutions in the embodiments of the present application more clearly, brief descriptions of the accompanying drawings required for describing the embodiments are provided below. Apparently, the accompanying drawings in the following description show only some embodiments of the present application, and those skilled in the art can obtain other drawings based on these accompanying drawings without creative efforts. For a more complete understanding of the present application and its beneficial effects, the following description is provided in conjunction with the accompanying drawings, where the same reference numerals in the following description denote the same components.

FIG. 1 is a schematic diagram of a phototherapy device of the present application.

FIG. 2 is an exploded view of the phototherapy device of the present application.

FIG. 3 is a partial sectional view of the phototherapy device of the present application.

FIG. 4 is a schematic diagram of a phototherapy component of the phototherapy device of the present application.

FIG. 5 is a sectional view of a first docking member and a button of the phototherapy device of the present application.

FIG. 6 is another state schematic diagram of the phototherapy component of the phototherapy device of the present application.

FIG. 7 is a schematic diagram of a support base of the phototherapy device of the present application.

FIG. 8 is a schematic diagram of a support main body of the phototherapy device of the present application.

FIG. 9 is a schematic diagram of a deformable member of the phototherapy device of the present application.

FIG. 10 is a schematic diagram of a second docking member of the phototherapy device of the present application.

### REFERENCE NUMERALS

100, phototherapy device;
10, phototherapy component; 10a, containing groove; 10b, moving groove; 11, phototherapy module; 12, mounting module; 121, first docking member; 1211, first docking portion; 1212, first inclined portion; 1213, snap-fit portion; 13, button; 131, moving main body; 132, pressing portion; 133, second inclined portion; 14, phototherapy main body; 14a, through hole; 15, connection belt;
20, support base; 21, support main body; 211, base; 212, lifting portion; 213, tightening member; 22, deformable member; 221, deformable main body; 222, swing member; 23, second docking member; 23a, rotation hole; 231, second docking portion.

### DETAILED DESCRIPTIONS OF EMBODIMENTS

The technical solutions in the embodiments of the present application will be clearly and completely described below with reference to the accompanying drawings in the embodiments of the present application. Obviously, the described embodiments are only some rather than all of the embodiments of the present application. Based on the embodiments in the present application, all other embodiments obtained by those skilled in the art without creative efforts shall fall within the scope of protection of the present application.

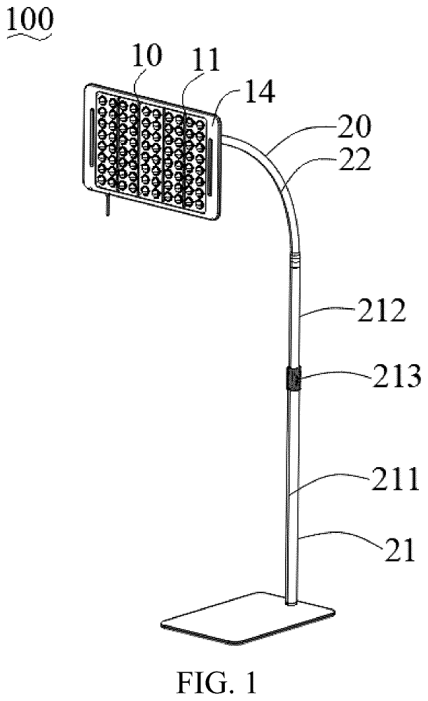

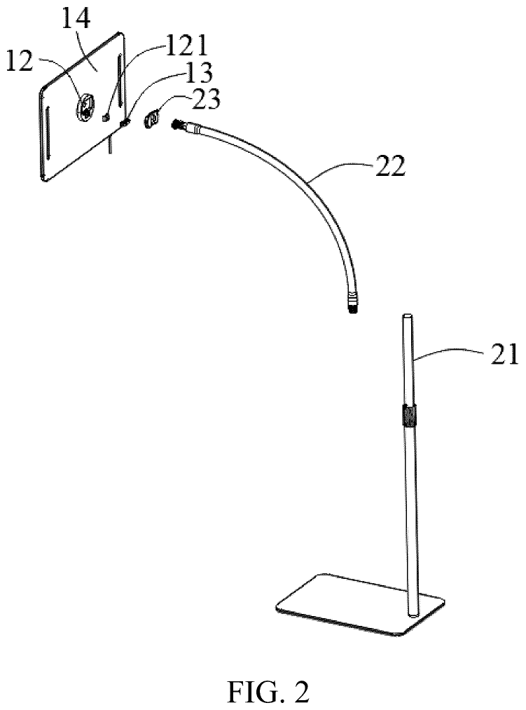

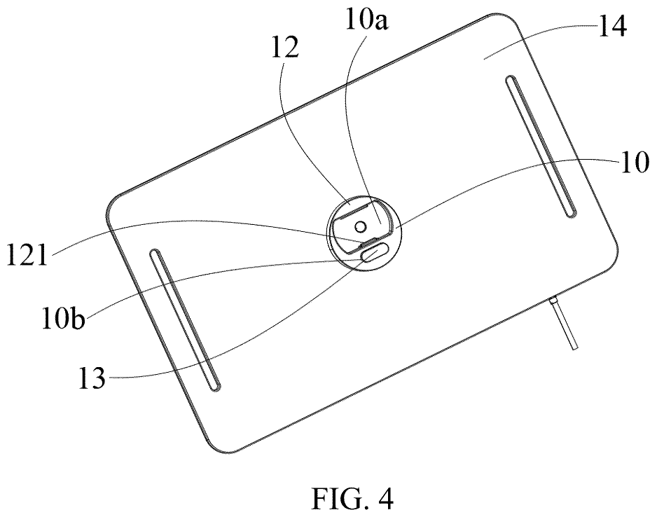

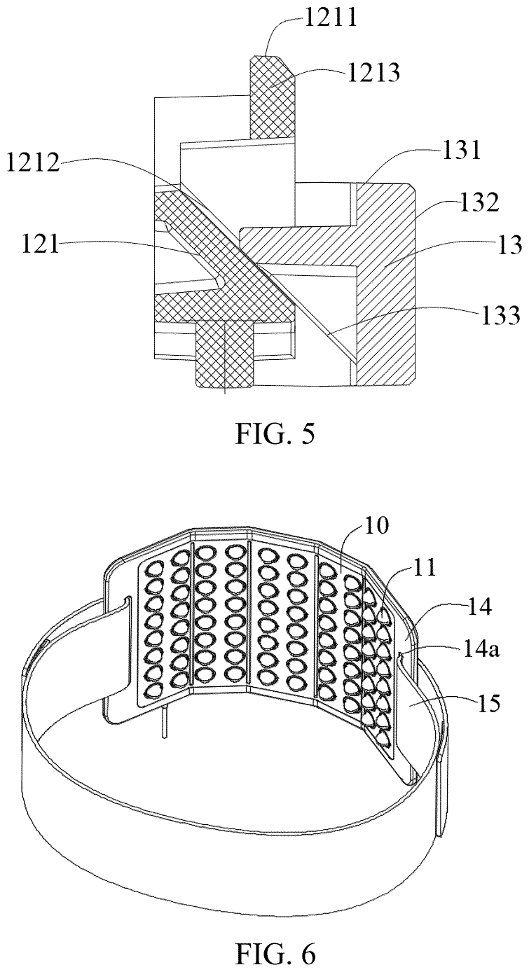

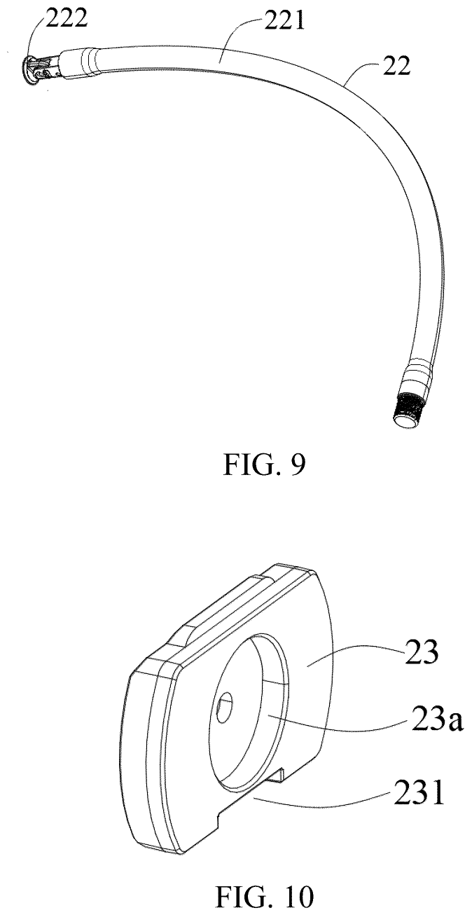

Referring to FIGS. 1 to 10, the present application provides a phototherapy device 100, which is configured to emit light to a user. Optionally, the light is red light, which has a physical therapy effect on the user's skin.

Referring to FIGS. 1 to 10, in the present application, the phototherapy device 100 includes a phototherapy component 10 and a support base 20. The phototherapy component 10 is provided with a phototherapy module 11 and a mounting module 12. The phototherapy module 11 is provided on a front surface of the phototherapy component 10 for outputting corresponding phototherapy light. The mounting module 12 is provided on a rear surface of the phototherapy component 10 and provided with a first docking member 121. The support base 20 is provided with a support main body 21, a deformable member 22 and a second docking member 23. The deformable member 22 is connected to the support main body 21 and can deform under external force. The second docking member 23 is connected to the end of the deformable member 22 away from the support main body 21 and can rotate relative to the end of the deformable member 22 away from the support main body 21. The second docking member 23 is detachably docked with the first docking member 121, and the phototherapy component 10 can rotate along with the rotation of the second docking member 23. Thus, the phototherapy component 10 can be deformed through the deformable member 22, meanwhile, the phototherapy component 10 can adjust its position in different directions during rotation, enabling compatibility with multiple different scenarios, thereby avoiding that the phototherapy component can only maintain a single state without deformation capability, and improving the scenario compatibility of the phototherapy device 100.

Referring to FIGS. 1 to 10, in the present application, the phototherapy component 10 is provided with the phototherapy module 11 and the mounting module 12. The phototherapy module 11 is provided on the front surface of the phototherapy component 10 for outputting corresponding phototherapy light, so that the phototherapy module 11 can emit the corresponding phototherapy light toward the user from the front side to facilitate the user to receive phototherapy. The mounting module 12 is provided on the rear surface of the phototherapy component 10 and provided with the first docking member 121, and the first docking member 121 is located on the rear side wall of the mounting module 12.

The support base 20 is provided with the support main body 21, the deformable member 22 and the second docking member 23. The deformable member 22 is connected to the support main body 21 and can deform under external force. The second docking member 23 is connected to the end of the deformable member 22 away from the support main body 21 and can rotate relative to the end of the deformable member 22 away from the support main body 21, so as to adjust the rotation angle of the second docking member 23 relative to the deformable member 22. The second docking member 23 is detachably docked with the first docking member 121, so that the second docking member 23 can be connected to or disconnected from the first docking member 121, thereby enabling the mounting module 12 to be detached from the support base 20 and improving the assembly and disassembly convenience of the phototherapy component 10. In this case, the phototherapy component 10 can rotate along with the rotation of the second docking member 23, so that the phototherapy component 10 can be deformed through the deformable member 22; meanwhile, the phototherapy component 10 can adjust its position in different directions during rotation, enabling compatibility with multiple different scenarios, thereby avoiding that the phototherapy component can only maintain a single state without deformation capability, and improving the scenario compatibility of the phototherapy device 100.

Referring to FIGS. 1 to 3, in the present application, the first docking member 121 is provided with a first docking portion 1211. The second docking member 23 is provided with a second docking portion 231, the second docking portion 231 is provided opposite to the first docking portion 1211, and the second docking portion 231 is in snap-fit with the first docking portion 1211. Thus, the first docking member 121 can be detachably connected to the second docking member 23 through that the first docking portion 1211 is in snap-fit with the second docking portion 231.

A rotatable structure is formed between the end of the deformable member 22 away from the support main body 21 and the second docking member 23, and the second docking member 23 rotates relative to the end of the deformable member 22 away from the support main body 21, so as to adjust a rotation angle of the second docking member 23 relative to the deformable member 22.

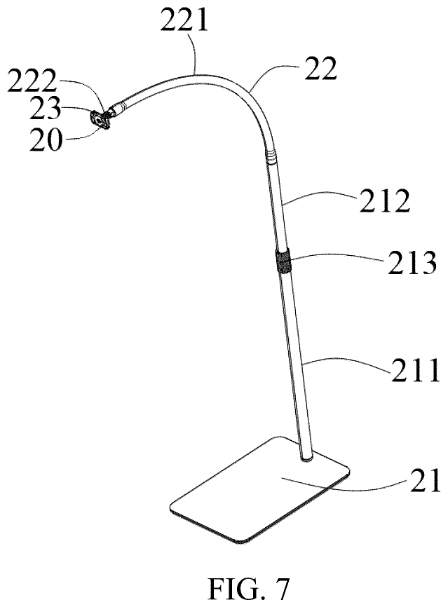

Referring to FIGS. 7 to 10, in the present application, the second docking member 23 is provided with a rotation hole 23a; the end of the deformable member 22 away from the support main body 21 is inserted into the rotation hole 23a, so that the second docking member 23 can rotate relative to the deformable member 22 through the rotation hole 23a. In this case, the second docking member 23 can rotate along an axis of the rotation hole 23a and drive the rotation of the phototherapy component 10 through the first docking member 121, so that the phototherapy component 10 can rotate relative to the deformable member 22 through the first docking member 121 and the second docking member 23 under external force. Optionally, the rotation angle of the phototherapy component 10 ranges from 0° to 360°.

Referring to FIGS. 7 to 9, in the present application, the deformable member 22 is provided with a deformable main body 221 and a swing member 222. The deformable main body 221 is connected to the support main body 21 and located on one side of the support main body 21. The swing member 222 is swingably connected to the deformable main body 221 and horizontally swings, so as to adjust the swing angle of the swing member 222 relative to the deformable main body 221, thereby enabling the swing member 222 to swing left and right. The front surface of the swing member 222 is provided with the second docking member 23, and the swing member 222 is connected to the phototherapy component 10 through the second docking member 23 and the first docking member 121. The phototherapy component 10 adjusts its horizontal position along with the horizontal swinging of the swing member 222 relative to the deformable main body 221, so that the phototherapy component 10 can swing left and right through the swing member 222, ensuring that the phototherapy component 10 is compatible with left-facing or right-facing application scenarios. Optionally, the deformable main body 221 is a gooseneck tube.

Referring to FIG. 7, in the present application, the swing member 222 is hinged to the deformable main body 221 and horizontally swings along the hinged position, so that the swing member 222 can swing relative to the deformable main body 221 in a hinged manner. The deformable main body 221 is provided with a deformable tube inside, the deformable tube can deform under external force, and the outer surface of the deformable main body 221 is a colloid layer, so that the deformable main body 221 can be adjusted from a linear state to a bent state, thereby enabling the phototherapy component 10 to adjust the angle facing the user according to the bending angle of the deformable main body 221. This ensures that the phototherapy component 10 can emit light to different portions of the user when the user maintains the same posture, enabling compatibility with multiple different scenarios.

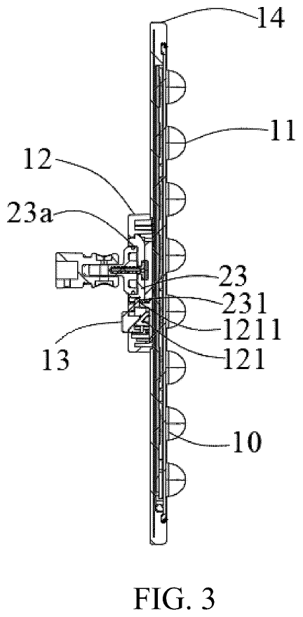

Referring to FIGS. 3 to 5, in the present application, the rear surface of the phototherapy component 10 is provided with a containing groove 10a. The first docking member 121 is provided in the vertical direction and is telescopically connected to the rear surface of the phototherapy component 10, and can telescope relative to the containing groove 10a, so as to adjust the vertical position of the first docking member 121 relative to the containing groove 10a. The second docking member 23 is inserted into the containing groove 10a and is in a docking state with the first docking member 121 inside the containing groove 10a. In this case, the first docking member 121 is inserted into a snap-fit groove of the second docking member 23 and is in snap-fit with the second docking member 23, so as to realize the detachable connection between the second docking member 23 and the first docking member 121. When the first docking member 121 contracts downward, the first docking member 121 is disengaged from the snap-fit groove of the second docking member 23, so that the second docking member 23 can be disconnected from the first docking member 121. When the first docking member 121 extends upward, the first docking member 121 is in snap-fit with the snap-fit groove of the second docking member 23, so that the second docking member 23 can be connected to the first docking member 121. Optionally, the snap-fit groove is recessed in the second docking member 23 from bottom to top.

Referring to FIGS. 3 to 5, in the present application, the rear surface of the phototherapy component 10 is provided with a button 13, the button 13 is movably connected to the phototherapy component 10 and can telescope relative to the rear surface of the phototherapy component 10, so as to adjust a front position and a rear position of the button 13 relative to the phototherapy component 10. The button 13 is connected to the first docking member 121 and drives the first docking member 121 to telescope relative to the containing groove 10a during movement, so that the button 13 can control the telescoping of the first docking member 121, thereby enabling the phototherapy component 10 to be detached by pressing the button 13.

The button 13 is elastically movable relative to the phototherapy component 10 by means of a spring, and the spring exerts an elastic force on the button 13. When no external force is applied to the button 13, the button 13 is protruded from the rear surface of the phototherapy component 10, and the first docking member 121 is in an extended state relative to the containing groove 10a, so that the first docking member 121 can be in snap-fit with the snap-fit groove of the second docking member 23. When the button 13 is pressed under external force, the button 13 contracts into the rear surface of the phototherapy component 10, and the first docking member 121 is in a contracted state relative to the containing groove 10a, so that the first docking member 121 can be disengaged from the snap-fit groove of the second docking member 23.

Referring to FIGS. 3 to 5, in the present application, the rear surface of the phototherapy component 10 is provided with a moving groove 10b. The first docking member 121 is provided with a first inclined portion 1212 and a snap-fit portion 1213, and the first inclined portion 1212 and the snap-fit portion 1213 are a unitary one-piece structure. The button 13 is provided with a moving main body 131, a pressing portion 132 and a second inclined portion 133. The moving main body 131 is movably connected to the moving groove 10b, so as to adjust the position of the moving main body 131 relative to the moving groove 10b, thereby enabling the moving main body 131 to move relative to the phototherapy component 10 through the moving groove 10b. The pressing portion 132 is connected to the moving main body 131 and located on the rear side wall of the button 13, and the pressing portion 132 serves as the pressing position of the button 13. The second inclined portion 133 is connected to the moving main body 131 and docked with the first inclined portion 1212. The first inclined portion 1212 contacts different positions of the second inclined portion 133 during the movement of the second inclined portion 133, and drives the snap-fit portion 1213 to telescope relative to the containing groove 10a, so that the snap-fit portion 1213 is in snap-fit with or disengaged from the second docking member 23. Through the movement of the button 13 and the double-inclined-plane transmission, the snap-fit portion 1213 is telescoped to realize one-press unlocking and one-insertion locking for quick assembly and disassembly of the phototherapy component 10, respectively, which improves the assembly and disassembly convenience of the phototherapy component 10 and facilitates the separate use of the phototherapy component 10.

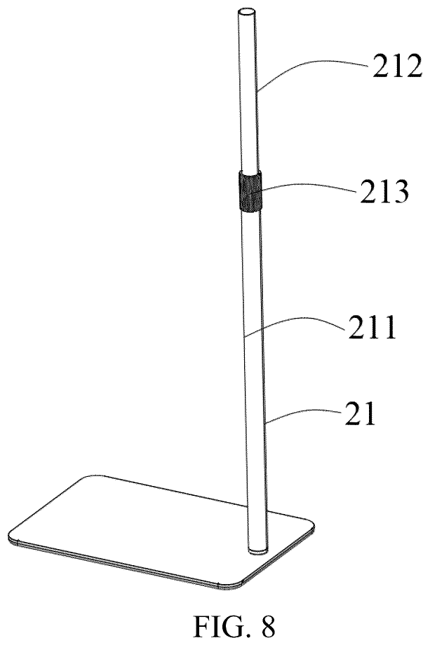

Referring to FIG. 8, in the present application, the support main body 21 is provided with a base 211 and a lifting portion 212. The base 211 is used for being laid on the ground, so that the phototherapy component 10 can be lifted as the lifting portion 212 is lifted, thereby enabling the phototherapy component 10 to be lifted as the deformable member 22 is lifted to adjust the height position of the phototherapy component 10, so as to adapt to scenarios with different heights.

The lifting portion 212 penetrates through the base 211, lifts relative to the base 211 along the vertical direction, and is fixed on the base 211 through a tightening member 213 to maintain a corresponding height. When the tightening member 213 is rotated under external force, the tightening member 213 can lock or unlock the lifting portion 212 and the base 211. When the tightening member 213 unlocks the lifting portion 212 and the base 211, the lifting portion 212 can be adjusted in height relative to the base 211. When the tightening member 213 locks the lifting portion 212 and the base 211, the lifting portion 212 is in a fixed state relative to the base 211, which prevents the lifting portion 212 from being accidentally lifted or lowered relative to the base 211, so that the phototherapy component 10 can maintain the corresponding height.

Referring to FIG. 6, in the present application, the phototherapy component 10 is provided with a phototherapy main body 14. The phototherapy main body 14 supports the phototherapy module 11 and the mounting module 12, so that the phototherapy module 11 and the mounting module 12 are fixed on the phototherapy main body 14. A plurality of phototherapy modules 11 are provided along the length direction of the phototherapy main body 14. Two adjacent phototherapy modules 11 are in a planar state or an angled state, so that the phototherapy component 10 can fit any curved surface of the user's body portion like a flexible piece. Optionally, there are 5 phototherapy modules 11.

The phototherapy main body 14 is provided with a plurality of through holes 14a; each through hole 14a is used for a connection belt 15 to pass through, and the connection belt 15 passes through the corresponding through hole 14a, so that the connection belt 15 can be connected to the phototherapy main body 14 through the plurality of through holes 14a. The connection belt 15 can be wound around the user's body portion, so that the phototherapy main body 14 can be fixed on the user's body portion through the connection belt 15, avoiding the user supporting the phototherapy main body 14 with hands. This ensures that the light emitted by the phototherapy module 11 is always closely attached to the user's skin without displacement or omission, realizing a wearable phototherapy component 10.

Compared with the related art, the beneficial effects of the present application are as follows:

The present application provides a phototherapy device 100. The phototherapy component 10 is provided with a phototherapy module 11 and a mounting module 12. The phototherapy module 11 is provided on a front surface of the phototherapy component 10 for outputting corresponding phototherapy light. The mounting module 12 is provided on a rear surface of the phototherapy component 10 and provided with a first docking member 121. The support base 20 is provided with a support main body 21, a deformable member 22 and a second docking member 23. The deformable member 22 is connected to the support main body 21 and can deform under external force. The second docking member 23 is connected to the end of the deformable member 22 away from the support main body 21 and can rotate relative to the end of the deformable member 22 away from the support main body 21. The second docking member 23 is detachably docked with the first docking member 121, at this time, the phototherapy component 10 can rotate along with the rotation of the second docking member 23. Thus, the phototherapy component 10 can be deformed through the deformable member 22, meanwhile, the phototherapy component 10 can adjust its position in different directions during rotation, enabling compatibility with multiple different scenarios, thereby avoiding that the phototherapy component can only maintain a single state without deformation capability, and improving the scenario compatibility of the phototherapy device 100.

In the above embodiments, the description of each embodiment has its own emphasis. For portions not described in detail in a certain embodiment, reference may be made to the relevant descriptions of other embodiments. In the description of the present application, the terms “first” and “second” are used for descriptive purposes only, and shall not be construed as indicating or implying relative importance or implicitly indicating the number of the indicated technical features. Thus, a feature defined with “first” or “second” may explicitly or implicitly include one or more such features.

Specific examples are used herein to illustrate the principles and implementations of the present application. The description of the above embodiments is only intended to help understand the method and core idea of the present application. Meanwhile, for those skilled in the art, according to the idea of the present application, there will be changes in the specific implementations and application scopes. In conclusion, the content of this specification shall not be construed as a limitation to the present application.

What is claimed is:

1. A phototherapy device, comprising:
a phototherapy component, provided with a phototherapy module and a mounting module; and
a support base, provided with a support main body, a deformable member and a second docking member;
wherein the phototherapy module is provided on a front surface of the phototherapy component for outputting corresponding phototherapy light;
the mounting module is provided on a rear surface of the phototherapy component and provided with a first docking member;
the deformable member is connected to the support main body and is deformable under external force;
the second docking member is connected to and is rotatable relative to an end of the deformable member away from the support main body; and
the second docking member is detachably docked with the first docking member, and the phototherapy component is rotatable along with a rotation of the second docking member.

2. The phototherapy device according to claim 1, wherein the first docking member is provided with a first docking portion;
the second docking member is provided with a second docking portion, and the second docking portion is snap-fit with the first docking portion; and
the second docking member is rotatable relative to the end of the deformable member away from the support main body.

3. The phototherapy device according to claim 2, wherein the second docking member is provided with a rotation hole;
the end of the deformable member away from the support main body is inserted into the rotation hole, and the second docking member is rotatable along an axis of the rotation hole and is configured to drive the phototherapy component to rotate through the first docking member.

4. The phototherapy device according to claim 2, wherein the deformable member is provided with a deformable main body and a swing member;
the deformable main body is connected to the support main body and located on one side of the support main body;
the swing member is swingably connected to the deformable main body and is configured to swing horizontally;
a front surface of the swing member is provided with the second docking member, and the swing member is connected to the phototherapy component through the second docking member and the first docking member; and
a horizontal position of the phototherapy component is adjusted along with a horizontal swinging of the swing member relative to the deformable main body.

5. The phototherapy device according to claim 4, wherein the swing member is hinged to the deformable main body and is configured to swing horizontally along a hinged position; and
the deformable main body is provided with a deformable tube inside, the deformable tube is deformable under external force, and an outer surface of the deformable main body is a colloid layer.

6. The phototherapy device according to claim 2, wherein the rear surface of the phototherapy component is provided with a containing groove;
the first docking member is telescopically connected to the rear surface of the phototherapy component and is telescopical relative to the containing groove;
the second docking member is inserted into the containing groove and is in a docking state with the first docking member in the containing groove; and
the second docking member is inserted into a snap-fit groove of the first docking member and is in snap-fit with the first docking member.

7. The phototherapy device according to claim 6, wherein the rear surface of the phototherapy component is provided with a button;
the button is movably connected to the phototherapy component and is telescopical relative to the rear surface of the phototherapy component; and
the button is connected to the first docking member and is configured to drive the first docking member to telescope relative to the containing groove during movement.

8. The phototherapy device according to claim 7, wherein the rear surface of the phototherapy component is provided with a moving groove;
the first docking member is provided with a first inclined portion and a snap-fit portion, and the first inclined portion and the snap-fit portion are a unitary one-piece structure;
the button is provided with a moving main body, a pressing portion and a second inclined portion;
the moving main body is movably connected to the moving groove;
the pressing portion is connected to the moving main body and located on a rear side wall of the button, and the pressing portion serves as a pressing position of the button;
the second inclined portion is connected to the moving main body and docked with the first inclined portion; and
the first inclined portion is configured to contact different positions of the second inclined portion during a movement of the second inclined portion, and drive the snap-fit portion to telescope relative to the containing groove, so that the snap-fit portion is in snap-fit with or separated from the second docking member.

9. The phototherapy device according to claim 1, wherein the support main body is provided with a base and a lifting portion;
the base is configured for being laid on the ground;
the lifting portion is liftably connected to the base and is configured for supporting the deformable member; and
the lifting portion is configured to penetrate through the base, lift relative to the base along a vertical direction, and is fixed on the base through a tightening member to maintain a corresponding height.

10. The phototherapy device according to claim 1, wherein the phototherapy component is provided with a phototherapy main body which supports the phototherapy module and the mounting module;
a plurality of phototherapy modules are provided along a length direction of the phototherapy main body; and
two adjacent phototherapy modules are in a planar state or an angled state.

11. The phototherapy device according to claim 10, wherein the phototherapy main body is provided with a plurality of through holes;
each through hole is configured for a connection belt to pass through, and the connection belt is configured to pass through a corresponding through hole and wound around a user's body portion.

12. A phototherapy device, comprising:
a phototherapy component, provided with a phototherapy module and a mounting module;
a support base, provided with a support main body, a deformable member and a second docking member;
wherein the phototherapy module is provided on a front surface of the phototherapy component for outputting corresponding phototherapy light;
the mounting module is provided on a rear surface of the phototherapy component and provided with a first docking member;
the deformable member is connected to the support main body and is deformable under external force; and
the second docking member is connected to an end of the deformable member away from the support main body.

13. The phototherapy device according to claim 12, wherein the deformable member is provided with a deformable main body and a swing member;
the deformable main body is connected to the support main body and located on one side of the support main body;
the swing member is swingably connected to the deformable main body and is configured to swing horizontally;
a front surface of the swing member is provided with the second docking member, and the swing member is connected to the phototherapy component through the second docking member and the first docking member; and
a horizontal position of the phototherapy component is adjusted along with a horizontal swinging of the swing member relative to the deformable main body.

14. The phototherapy device according to claim 13, wherein the swing member is hinged to the deformable main body and is configured to swing horizontally along a hinged position.

15. The phototherapy device according to claim 12, wherein the deformable main body is provided with a deformable tube inside, the deformable tube is deformable under external force, and an outer surface of the deformable main body is a colloid layer.

16. The phototherapy device according to claim 12, wherein the rear surface of the phototherapy component is provided with a containing groove;
the first docking member is telescopically connected to the rear surface of the phototherapy component and is telescopical relative to the containing groove;
the second docking member is inserted into the containing groove and is in a docking state with the first docking member in the containing groove; and
the first docking member is inserted into a snap-fit groove of the second docking member and is in snap-fit with the second docking member.

17. The phototherapy device according to claim 16, wherein the rear surface of the phototherapy component is provided with a button;
the button is movably connected to the phototherapy component and is telescopical relative to the rear surface of the phototherapy component; and
the button is connected to the first docking member and is configured to drive the first docking member to telescope relative to the containing groove during movement.

18. The phototherapy device according to claim 12, wherein the second docking member is rotatable relative to the end of the deformable member away from the support main body;
the second docking member is docked with the first docking member, and the phototherapy component is rotatable along with a rotation of the second docking member;
the first docking member is provided with a first docking portion;
the second docking member is provided with a second docking portion, and the second docking portion is in snap-fit with the first docking portion;
a rotatable structure is formed between the end of the deformable member away from the support main body and the second docking member; and
the second docking member is rotatable relative to the end of the deformable member away from the support main body.

19. The phototherapy device according to claim 18, wherein the second docking member is provided with a rotation hole;
the end of the deformable member away from the support main body is inserted into the rotation hole, and the second docking member is rotatable along an axis of the rotation hole and is configured to drive a rotation of the phototherapy component through the first docking member.

20. A phototherapy device, comprising:
a phototherapy component, provided with a phototherapy module and a mounting module; and
a support base, provided with a support main body, a deformable member and a second docking member;
wherein the phototherapy module is provided on a front surface of the phototherapy component and configured for outputting corresponding phototherapy light;
the mounting module is provided on a rear surface of the phototherapy component and provided with a first docking member;
the deformable member is connected to the support main body and is deformable under external force;
the second docking member is connected to an end of the deformable member away from the support main body;
the second docking member is docked with the first docking member;
the support main body is provided with a base and a lifting portion;
the base is configured for being laid on the ground;
the lifting portion is liftably connected to the base and is configured to support the deformable member; and
the lifting portion is configured to penetrate through the base, lift relative to the base along a vertical direction, and is fixed on the base through a tightening member to maintain a corresponding height.
